(19) United States
(12) Patent Application Publication
(10) Pub. No.: US 2009/0088610 A1
(43) Pub. Date: Apr. 2, 2009

(54) Measuring Physiological Response to Media for Viewership Modeling

(76) Inventors: Hans C. Lee, Carmel, CA (US); Michael J. Lee, Carmel, CA (US); Tim Hong, San Diego, CA (US); William H. Williams, Hilo, HI (US)

Correspondence Address: COURTNEY STANIFORD & GREGORY LLP, P.O. BOX 9686, SAN JOSE, CA, 95157, US

(21) Appl. No.: 12/206,702

(22) Filed: Sep. 8, 2008

Related U.S. Application Data
(63) Continuation-in-part of application No. 11/681,265, filed on Mar. 2, 2007.
Application No. 11/681,265 is a continuation-in-part of application No. 11/804,517, filed on May 17, 2007.
(60) Provisional application No. 60/970,898, filed on Sep. 7, 2007.
(60) Provisional application No. 60/970,900, filed on Sep. 7, 2007.
(60) Provisional application No. 60/970,905, filed on Sep. 7, 2007.
(60) Provisional application No. 60/970,908, filed on Sep. 7, 2007.
(60) Provisional application No. 60/970,913, filed on Sep. 7, 2007.
(60) Provisional application No. 60/970,916, filed on Sep. 7, 2007.
(60) Provisional application No. 60/970,920, filed on Sep. 7, 2007.

(51) Int. Cl. A61B 5/00

(52) U.S. Cl. 600/301

(57) ABSTRACT

Embodiments of a system to accurately record a viewer's emotion as he or she is watching or listening to media content are described. The system records physiological data from viewers and transmits it back to a central location as viewers watch, listen to, or otherwise interact with media such as TV, radio, video games, web sites or other media. An integrated physiological sensing device measures viewers' cognitive and emotional responses to media and transmits to a base station in close proximity to the sensing device. The physiological sensing base station can be integrated into home electronics devices such as digital video recorders, TV cable boxes, video cassette recorders, DVD players and gaming systems to record viewer's emotional and cognitive responses to the media and combining this response with context data from the electronics equipment to know what the viewer is responding to.

### CROSS-REFERENCE TO RELATED APPLICATIONS

This application is a continuation in part application of U.S. patent application Ser. No. 11/681,265, filed Mar. 2, 2007.

This application is a continuation in part application of U.S. patent application Ser. No. 11/804,517, filed May 17, 2007.

This application claims the benefit of U.S. patent application No. 60/970,898, filed Sep. 7, 2007.

This application claims the benefit of U.S. patent application No. 60/970,900, filed Sep. 7, 2007.

This application claims the benefit of U.S. patent application No. 60/970,905, filed Sep. 7, 2007.

This application claims the benefit of U.S. patent application No. 60/970,908, filed Sep. 7, 2007.

This application claims the benefit of U.S. patent application No. 60/970,913, filed Sep. 7, 2007.

The present application claims the benefit of the U.S. Provisional Application No. 60/970,916 entitled “Methods and Systems for Media Viewer Attention Detection Using Means for Improving Information About Viewer's Preferences, Media Viewing Habits, and Other Factors,” and filed on Sep. 7, 2007.

The present application claims the benefit of the U.S. Provisional Application No. 60/970,920 entitled “Measuring Physiological Response to Media for Viewership Modeling By Integrating Into Home Electronics,” and filed on Sep. 7, 2007.

### FIELD

Embodiments of the invention relate generally to media distribution, and more specifically, to modeling viewership characteristics based on physiological data obtained from viewers.

### BACKGROUND

Display devices, such as televisions, computer monitors, personal digital devices, and the like are the principal means of delivering electronic content. Content providers can deliver virtually any type of visual content through a myriad number of display devices. The most common display means has traditionally been the television, however, the advent of the Internet and other networks has led to an increase in viewing through computers, game device, and other media playback units. Although certain user activity can be tracked and measured with regard to content delivery, such as network sites visited or television shows tuned into, there is no present way of knowing whether a person is actually viewing, reading, or otherwise perceiving what is displayed, when a television or computer monitor is turned on. Moreover, such systems have no way of indicating how a particular viewer feels about the content.

In general, emotions are a key indicator of how well viewers like or dislike a particular media item, and if they will likely want to watch it in again in the future. Such information is not currently available, except for experiments involving a small number of people in a laboratory setting.

One key issue with present systems is that they typically do not adequately measure an effectively wide enough range of emotions. A second issue with present systems is that even if physiological data that reflects emotions can be recorded, it is generally not converted to a usable form. Such data is not generally obtained for very large numbers of people in their natural settings, such as sitting at home while watching television or working on a computer. Therefore, such data cannot be made available to agencies or entities that can utilize this emotional response data.

### INCORPORATION BY REFERENCE

Each patent, patent application, and/or publication mentioned in this specification is herein incorporated by reference in its entirety to the same extent as if each individual patent, patent application, and/or publication was specifically and individually indicated to be incorporated by reference.

### BRIEF DESCRIPTION OF THE DRAWINGS

Embodiments of the present invention are illustrated by way of example and not limitation in the figures of the accompanying drawings, in which like references indicate similar elements and in which:

FIG. 1 illustrates a viewer physiological measurement system, under an embodiment.

FIG. 2 illustrates example of some of the characteristics for use by the sensors of FIG. 1, under an embodiment.

FIG. 3 illustrates an aggregate trace profile for use by the physiological sensing process, under an embodiment.

### DETAILED DESCRIPTION

Embodiments of a system to accurately record a viewer's emotion as he or she is watching or listening to media content are described. The system records physiological data from viewers and transmits it back to a central location as viewers watch, listen to, or otherwise interact with media such as TV, radio, video games, web sites or other media. An integrated physiological sensing device measures viewers' cognitive and emotional responses to media and transmits to a base station in close proximity to the sensing device. The physiological sensing base station can be integrated into home electronics devices such as digital video recorders, TV cable boxes, video cassette recorders, DVD players and gaming systems to record viewer's emotional and cognitive responses to the media and combining this response with context data from the electronics equipment to know what the viewer is responding to. Such a system allows data to be recorded for a very large number of people simultaneously as they sit in front of their televisions or computers.

Viewers react emotionally and rationally to media content. Initial reactions are emotional as viewers rationalize their reactions to media messages and then act on them emotionally. Understanding these reactions and how they impact thoughts and feelings about the message can be critical to creating effective and useful media content. In one embodiment, a physiological response measurement system incorporating a scalable physiological and brainwave measurement technology provides accurate, objective, and moment-by-moment analysis of how a viewer responds emotionally and cognitively to media messaging.

In one embodiment, one or more physiological sensors are attached to a viewer. The sensors measure certain physiological characteristics of the viewer that are relevant to emotional state while the viewer is perceiving media content. The signals recorded for the viewer can be for heart rate, EEG (electroencephalography measurements), EKG (electrocardiogram measurements), BVP, motion, position, temperature, galvanic skin response or other physiological indicators and are can be measured through sensors placed on or near the viewer. The sensor may include a set of electrodes or any appropriate fitting that attach to one or more portions of the viewer's body. In one embodiment, the sensors are provided in a sensor assembly or headset unit that attaches to the viewer's head to obtain brain wave measurements.

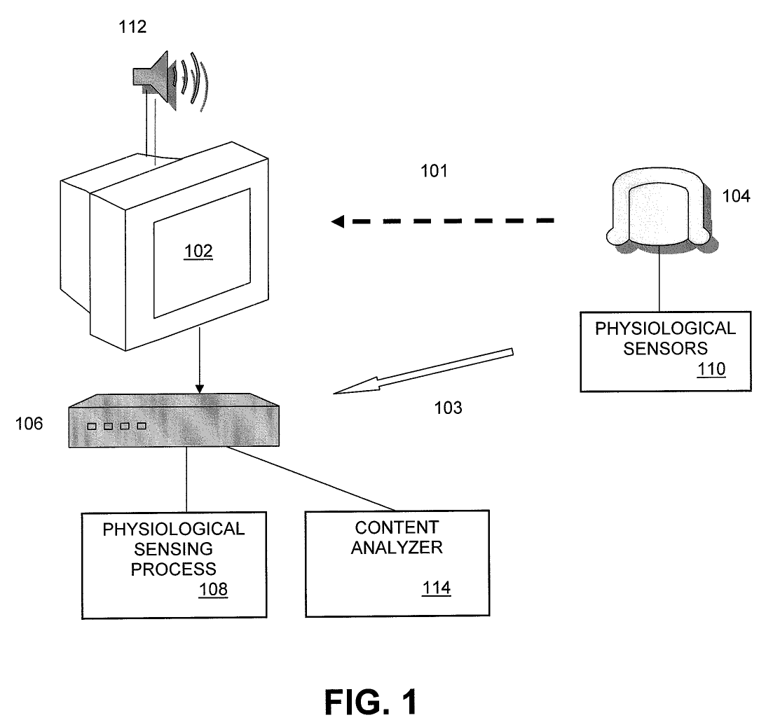

FIG. 1 illustrates a viewer physiological measurement system, under an embodiment. As shown in FIG. 1, a media delivery device (or “monitor”) 102 comprises a display device configured to display any type of visual content, such as streaming video, still pictures, or any other visually perceivable image in analog or digital format. The media delivery device 102 may be embodied in a television, computer monitor, electronic tablet, or any other electronic display device. An audio playback unit, such as speaker 112 may be coupled to or incorporated in the media delivery device to provide audio output for analog or digital sound signals. A viewer 104 is positioned to perceive the video and/or audio signals from the media delivery device 102.

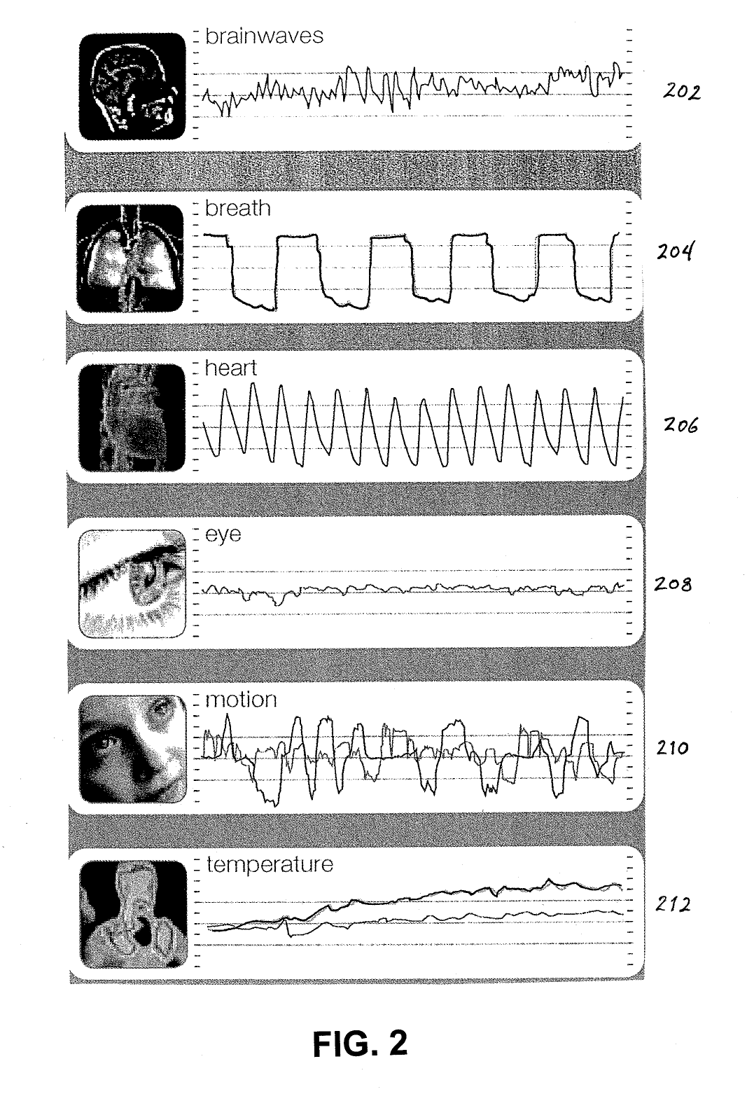

For the embodiment of FIG. 1, the viewer has one or more physiological sensors 110 attached to or in proximity to appropriate parts of the user's body, such as his or her head 104. The sensors are configured to measure relevant physiological characteristics of the viewer. Various different physiological characteristics may be measured. FIG. 2 illustrates example of some of the characteristics for use by the sensors of FIG. 1. As shown in FIG. 2, the sensor or sensors can be configured to measure brainwave activity 202, breath 204, heartbeat 206, eye movement 208, body motion 210, temperature 212, and any other relevant measurement.

As a user views, listens to or otherwise perceives 101 content provided by media delivery device 102 he or she registers appropriate physiological responses that are picked up by the sensors 110. This information is transmitted back to a central process so that viewer's physiological responses can be aggregated to create models of emotional and cognitive engagement in response to media.

In one embodiment, the central process comprises a physiological sensing process that is executed in a device 106 coupled to the media delivery device 102. For this embodiment, device 106 includes a processor and receiver to receive data from the external physiological sensing device 110. Transmission over line 103 can be done with a wired or wireless receiver that interfaces with the physiological sensing rig or sensor assembly.

The physiological sensing process 108 interprets the information received from the physiological sensors and then creates a packet of information that can be electronically sent out over the internet, over telephone lines or through other means to a central location(s) for use by other entities or agencies.

In one embodiment, the physiological sensing process 108 can be integrated within a media playback unit or source device that is closely coupled to or even incorporated within media delivery device 102. Such a device can be a home electronics device such as a digital video recorders, cable box, video cassette recorders, DVD player, gaming system, or any similar device.

In an alternative embodiment, the device 108 may be a separate device to playback device 108. Such a device records the media that the viewer is watching and also the physiological data and then sends both back to a central processing location. The media can then be analyzed to define what exactly the viewer was watching based on the sounds and visual content the viewer saw.

In one embodiment, the physiological information can be transmitted back over the cable over which the television signal was sent, to a central location. This can also be done using a telephone line, DSL (digital subscriber line) or wireless connection. The physiological data for each viewer is put into an electronic “packet” along with data about the media and sent to a new location that aggregates the information.

It should be noted that any of the connections between the components in any of FIG. 1 may be implemented through wired or wireless communication means. Likewise, in certain implementations, a computer-based network may be used to transmit one or more signals or data among the components.

A content analyzer component 114 may also be provided to analyze the audio/video content to extract meaningful information such as the media title (e.g., song name or movie name), where the content on a digital video recorder is, which radio station or television station is playing, and so on.

In this case, the physiological signals are tagged with a marker that defines which media segment the response corresponds to, such as a TV show, time-code, channel number, commercial, movie, video game segment/position, radio station, song, seconds into a show, recorded versus live on TV, etc. This data can then be interpreted to define emotional and cognitive responses second-by-second in correspondence with the media content.

Besides physiological data, certain user profile information can also be used. User profile information can include objective information about the viewer, such as age, gender, income and other indicators. This set of information can be sent back to a central location either back over the same TV cable, or over a telephone line or internet connection or other communication method. It can also be stored for later retrieval.

The disclosed system for integrating recorders for physiological sensors into home electronic devices is a key advance that enables physiological data to be recorded in home in a way it would never otherwise be able to be recorded. This replaces the arcane viewership modeling based solely on how many TVs or radios are on, with actual emotional response of viewers to each piece of media.

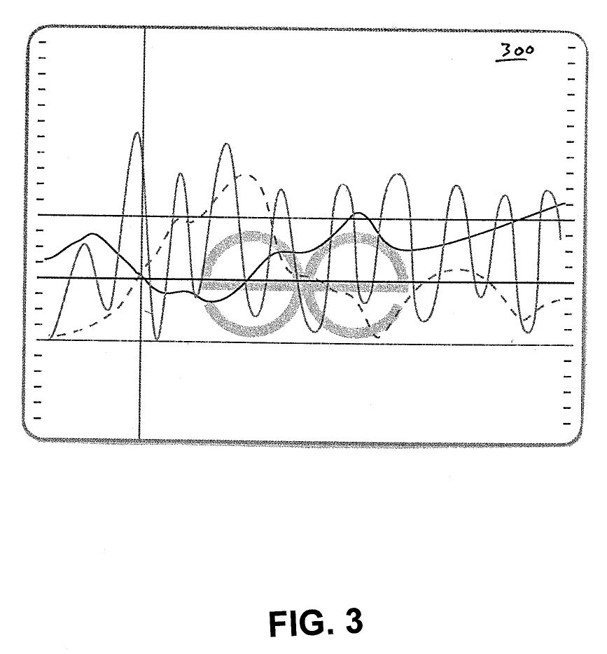

The ability to track and measure involuntary and unfiltered brainwave responses to media content can provide major tools to marketers who understand that consumers do not make decisions in a purely rational, linear fashion, and that emotion has a predominant impact on these responses. The sensor system provides a non-invasive tracking of certain body responses. The sensing process 108 provides non-biased assessment of media and message content. In one embodiment, the sensing process 108 provides an aggregate trace profile that provides a picture of the emotional response of the viewer. This profile can be a combination of any or all of the responses provided by the sensors and illustrated in FIG. 2. FIG. 3 illustrates an aggregate trace profile for use by the physiological sensing process, under an embodiment.

This information can then be used by content providers to model or predict viewership response to certain media content, and modify such content accordingly. Such content can comprise television shows, movies, songs, advertisements, video games, still pictures, spoken audio content, displayed text content (e.g., e-books and the like) or any other similar electronically distributed media content.

Aspects of the embodiments described herein may be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects of the method include: microcontrollers with memory (such as EEPROM), embedded microprocessors, firmware, software, etc. Furthermore, aspects of the described method may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various functions disclosed herein may be described using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, and so on).

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

The above description of illustrated embodiments is not intended to be exhaustive or to limit the embodiments to the precise form or instructions disclosed. While specific embodiments of, and examples for, the disclosed system are described herein for illustrative purposes, various equivalent modifications are possible within the scope of the described embodiments, as those skilled in the relevant art will recognize.

The elements and acts of the various embodiments described above can be combined to provide further embodiments. These and other changes can be made to the online loan application system in light of the above detailed description.

In general, in any following claims, the terms used should not be construed to limit the described system to the specific embodiments disclosed in the specification and the claims, but should be construed to include all operations or processes that operate under the claims. Accordingly, the described system is not limited by the disclosure, but instead the scope of the recited method is to be determined entirely by the claims.

While certain aspects of the system may be presented in certain claim forms, the inventor contemplates the various aspects of the methodology in any number of claim forms. For example, while only one aspect of the system is recited as embodied in machine-readable medium, other aspects may likewise be embodied in machine-readable medium. Accordingly, the inventor reserves the right to add additional claims after filing the application to pursue such additional claim forms for other aspects of the described systems and methods.

What is claimed is:

1. A system comprising:
a media delivery device configured to provide media content to a viewer;
a sensor assembly attached to one or more parts of a user's body, and configured to measure one or more physiological characteristics of the user while the viewer perceives the media content provided by the media delivery device;
a receiver circuit configured to receive data relating to the one or more physiological characteristics of the user; and
a physiological sensing process configured to interpret the physiological data to assess an emotional response of the viewer to the media content.

2. The system of claim 1 further comprising a content analyzer component configured to extract certain information from the media content, and to correlate specific portions of the media content with specific emotional responses.

3. The system of claim 2 further comprising a viewer profile component providing objective viewer profile information to supplement the emotional response.

4. The system of claim 1 wherein the physiological characteristics are selected from the group consisting of: brainwave activity, breath, heartbeat, eye movement, body motion, and temperature.

5. The system of claim 1 wherein the media delivery device is one of a television or a computer monitor.

6. The system of claim 1 wherein the sensor assembly comprises a head gear positioned on the head of the user and positioned to transmit the signal in the direction of the media delivery device.

7. The system of claim 1 wherein the physiological sensing process is integrated within a media playback device coupled to the media delivery device.

8. The system of claim 7 wherein the media playback device is selected from the group consisting of: a digital video recorder, a TV cable box, a video cassette recorder, DVD player, and a gaming system.
